SEQUENCE LISTING (25 sequences)

SEQ ID NO: 1 - DNA, 1159 bp, Homo sapiens
tctccacagtccttacattcaaaacgtttccctccaacctgacttttctgaacttcactgacagccacactgtggataaaggactcaccatactcatagaggttctctctagtatgcatgatctggtgctcaacaaattctgagatgacactgaacgacctcccacactcatcacatacatatggcattgccccaaaatcaattggctgtgactcggtaaaggagggggagctgaggctgctcaggctgctcacgctcatggcttttctcatctcactaccacattcaaagggcttcttcctgggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccaccacattttggtatgattcagcatcctgagatcgggactcataagccctggagtccctgtcgtcctctctgtccatttcaaagcttgttttcgccaccacaggaagggaaagatcccgcggaggcatcctgcttcttgggttcctggtgtgggaccagcggtctcgtggctccatgtctctgcttctgcccctccggtcccagtcccggtcaccactgaaagaatggactgagtgaggtggtgaggactctcttctgttccgggtcatgtcgtcgtcactggtcacgtcactgttgttgtcgtcttctggttggt

SEQ ID NO: 2 - DNA, 823 bp, Homo sapiens
tctccacagtccttacattcaaaacgtttccctccaacctgacttttctgaacttcactgacagccacactgtggataaaggactcaccatactcatagaggttctctctagtatgcatgatctggtgctcaacaaattctgagatgacactgaacgacctcccacactcatcacatacatatggcattgccccaaaatcaattggctgtgactcggtaaaggagggggagctgaggctgctcaggctgctcacgctcatggcttttctcatctcactaccacattcaaagggcttcttcctgggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtt

SEQ ID NO: 3 - DNA, 844 bp, Homo sapiens
tctccacagtccttacattcaaaacgtttccctccaacctgacttttctgaacttcactgacagccacactgtggataaaggactcaccatactcatagaggttctctctagtatgcatgatctggtgctcaacaaattctgagatgacactgaacgacctcccacactcatcacatacatatggcattgccccaaaatcaattggctgtgactcggtaaaggagggggagctgaggctgctcaggctgctcacgctcatggcttttctcatctcactaccacattcaaagggcttcttcctgggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtcca

SEQ ID NO: 4 - DNA, 846 bp, Homo sapiens
tctccacagtccttacattcaaaacgtttccctccaacctgacttttctgaacttcactgacagccacactgtggataaaggactcaccatactcatagaggttctctctagtatgcatgatctggtgctcaacaaattctgagatgacactgaacgacctcccacactcatcacatacatatggcattgccccaaaatcaattggctgtgactcggtaaaggagggggagctgaggctgctcaggctgctcacgctcatggcttttctcatctcactaccacattcaaagggcttcttcctgggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccacc

SEQ ID NO: 5 - DNA, 851 bp, Homo sapiens
tctccacagtccttacattcaaaacgtttccctccaacctgacttttctgaacttcactgacagccacactgtggataaaggactcaccatactcatagaggttctctctagtatgcatgatctggtgctcaacaaattctgagatgacactgaacgacctcccacactcatcacatacatatggcattgccccaaaatcaattggctgtgactcggtaaaggagggggagctgaggctgctcaggctgctcacgctcatggcttttctcatctcactaccacattcaaagggcttcttcctgggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccaccacatt

SEQ ID NO: 6 - DNA, 856 bp, Homo sapiens
ggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccaccacattttggtatgattcagcatcctgagatcgggactcataagccctggagtccctgtcgtcctctctgtccatttcaaagcttgttttcgccaccacaggaagggaaagatcccgcggaggcatcctgcttcttgggttcctggtgtgggaccagcggtctcgtggctccatgtctctgcttctgcccctccggtcccagtcccggtcaccactgaaagaatggactgagtgaggtggtgaggactctcttctgttccgggtcatgtcgtcgtcactggtcacgtcactgttgttgtcgtcttctggttggt

SEQ ID NO: 7 - DNA, 850 bp, Homo sapiens
cctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccaccacattttggtatgattcagcatcctgagatcgggactcataagccctggagtccctgccgtcctctctgtccatttcaaagcttgttttcgccaccacaggaagggaaagatcccgcggaggcatcctgcttcttgggttcctggtgtgggaccagcggtctcgtggctccatgtctctgcttctgcccctccggtcccagtcccggtcaccactgaaagaatggactgagtgaggtggtgaggactctcttctgttccgggtcatgtcgtcgtcactggtcacgtcactgttgttgtcgtcttctggttggt

SEQ ID NO: 8 - DNA, 848 bp, Homo sapiens
tttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccaccacattttggtatgattcagcatcctgagatcgggactcataagccctggagtccctgtcgtcctctctgtccatttcaaagcttgttttcgccaccacaggaagggaaagatcccgcggaggcatcctgcttcttgggttcctggtgtgggaccagcggtctcgtggctccatgtctctgcttctgcccctccggtcccagtcccggtcaccactgaaagaatggactgagtgaggtggtgaggactctcttctgttccgggtcatgtcgtcgtcactggtcacgtcactgttgttgtcgtcttctggttggt

SEQ ID NO: 9 - DNA, 4758 bp, Homo sapiens
cgggaggagaggtttgggaggcgcgggagatgtccaccctgggctggtggcgccgccgggcgccgggcgccatgagggtgcgctaggcggctgttcgtgcccgaggctgcgcagcactgaggtgagctttgccttcttgatcttccgtccttcttggagacgactggcgagaggaagagggactaggtccaaacgctaggtggctgggtccagccggagacccgcaccaaggaggagatcatcgagctcttggtccttgagcagtacctgaccatcatccctgaaaagctcaagccttgggtgcgagcaaaaaagccggagaactgtgagaagctcgtcactctgctggagaattacaaggagatgtaccaaccagaagacgacaacaacagtgacgtgaccagtgacgacgacatgacccggaacagaagagagtcctcaccacctcactcagtccattctttcagtggtgaccgggactgggaccggaggggcagaagcagagacatggagccacgagaccgctggtcccacaccaggaacccaagaagcaggatgcctccgcgggatctttcccttcctgtggtggcgaaaacaagctttgaaatggacagagaggacgacagggactccagggcttatgagtcccgatctcaggatgctgaatcataccaaaatgtggtggacctcgctgaggacaggaaacctcacaacacaatccaggacaacatggaaaactacaggaagctgctctccctcggagtgcagcttgctgaagacgatggccactcccacatgacgcagggccactcatcaagatccaagagaagtgcctacccaagcaccagtcgaggtctaaaaactatgcctgaagccaaaaaatcaacccaccggcgggggatttgtgaagatgaatcttcccacggagtgataatggaaaaattcatcaaggatgtgtcacgcagttccaaatcgggaagagcaagggagtcaagcgaccggtcacagagattccccagaatgtcagatgataactggaaggacatttcattgaacaagagggagtcagtgatccagcagcgggtttatgaagggaatgcatttaggggaggctttaggtttaattcaacccttgtttccagaaagagagttcttgaaagaaagaggcgctatcattttgacacagatgggaagggctcgattcacgatcaaaaaggctgtcccaggaagaagccctttgaatgtggtagtgagatgagaaaagccatgagcgtgagcagcctgagcagcctcagctccccctcctttaccgagtcacagccaattgattttggggcaatgccatatgtatgtgatgagtgtgggaggtcgttcagtgtcatctcagaatttgttgagcaccagatcatgcatactagagagaacctctatgagtatggtgagtcctttatccacagtgtggctgtcagtgaagttcagaaaagtcaggttggagggaaacgttttgaatgtaaggactgtggagagaccttcaataagagtgccgccttggctgaacatcggaagattcatgctagaggttatcttgtggaatgtaagaatcaggaatgtgaggaagccttcatgcctagccccacctttagtgagcttcagaaaatatatggcaaagacaaattctacgagtgcagggtgtgtaaggaaaccttccttcatagttctgccctgattgagcaccagaaaatccactttggggatgacaaagataatgagcgtgaacatgaacgtgaacgtgaacgtgagcgcggggaaacctttaggcccagcccagcccttaatgagtttcagaaaatgtatggtaaagagaaaatgtacgaatgtaaggtgtgtggggagactttccttcatagctcatccctgaaagaacatcagaaaatccatactagagggaacccatttgaaaacaagggtaaagtgtgtgaggaaacctttattcctggtcagtcccttaaaaggcgtcagaaaacttacaataaggagaagctctgtgactttacagatggccgggatgccttcatgcaaagctcagagctcagtgagcatcagaaaattcattctcgaaagaacctctttgaaggcagagggtatgagaaatctgtcattcatagtgggccattcactgaatctcagaagagtcatactataacaagacctcttgaaagtgatgaggacgaaaaggcgttcaccattagctctaacccctatgaaaaccagaagattcccactaaggaaaatgtctatgaggcaaaatcatatgagaggtctgttattcatagcttagcctctgtggaagctcagaaaagtcacagtgtagcagggcccagtaaaccaaaagtaatggcagagtctaccattcagagcttcgatgctatcaaccatcagagagttcgtgctggagggaacacctctgaaggaagggaatacagtaggtctgttatccatagcttagtggcttccaaacctccaagaagtcacaatggaaatgaattggtggaatctaatgagaagggagaatcctccatttatatctcagaccttaatgataagcgacagaagattcctgccagagagaacccttgtgaagggggcagtaagaatcgcaactatgaagactctgtcatacagagtgtattccgtgccaaacctcagaaaagtgttcctggagagggatctggtgagtttaagaaggatggcgaattctctgttcccagctcaaatgtccgtgaataccagaaggctcgtgctaaaaagaaatacattgagcataggagcaatgagacctctgtaattcactctctgccttttggtgaacaaacatttcgccctcgagggatgctctatgaatgtcaggagtgtggggagtgctttgctcatagctctgacctcactgagcaccagaagattcatgatagggagaagccctctggaagcagaaactatgaatggtctgtcattcgcagcttggcccctactgaccctcaaacaagttacgcccaagagcagtatgctaaagagcaagcgcggaacaaatgtaaggacttcagacaattttttgctaccagcgaagacctcaacacaaaccagaaaatctatgaccaagagaagtctcatggcgaggagtctcaaggcgagaatactgatggggaggagacccacagcgaggagacccatggtcaggagacaattgaagaccctgtcattcaaggctcagacatggaagaccctcagaaggatgaccctgatgacaaaatctatgaatgtgaggactgtggcctgggctttgtggatctcacagacctcacagaccatcagaaagtccacagcaggaagtgcctggttgacagtcgggagtacacacattctgtaattcacacccattccatcagcgagtatcagagagattacactggagagcagctgtatgaatgtccaaagtgtggggaatcttttattcatagctcattccttttcgagcatcagagaatccatgaacaagaccagttgtattccatgaaggggtgtgatgatggttttattgccctcttgcccatgaagccacggaggaatcgtgctgcagagaggaatcctgctcttgctgggtcggccattcgatgccttttgtgtggacaaggcttcattcatagctctgcccttaatgagcatatgagacttcatagggaagatgatttactggagcagagccagatggctgaggaagctatcattccaggcttagccctcactgagtttcagagaagtcagaccgaagagagactctttgaatgtgcagtctgtggagaatctttcgtcaacccagcagaacttgcagatcacgtaactgttcataagaatgagccctatgagtacgggtcctcctatactcacacctcatttcttactgagcccctcaaaggagctataccattctatgaatgcaaggattgtggtaagtcctttattcatagcacagtcctcactaaacataaggagcttcatctggaagaagaagaagaagatgaagcagcagcagctgcagcagcagcagcccaggaagttgaagccaatgtccatgttccacaagtagttctgaggattcagggcttaaacgtagaggctgctgagccagaagtggaggctgccgagccagaagtggaggctgctgagccagaagtggaggctgctgagccaaacggagaggctgaagggccagatggagaggctgcagagcccattggagaggctggacagccaaatggagaggccgagcagccaaatggggatgctgatgagccagatggtgcaggtattgaagacccagaagaaagagctgaagagccagagggaaaagctgaagagccagagggagatgccgacgagcctgacggtgtgggaattgaagacccagaagaaggtgaagatcaagagattcaggtagaagaaccatactatgactgccatgaatgcacagaaaccttcacttccagcacagcattcagtgaacacctgaaaactcatgccagcatgatcatatttgagcctgcaaatgcctttggggagtgctcaggctacatcgaacgtgccagcaccagcacaggtggtgccaatcaagctgatgagaagtacttcaaatgtgacgtctgtgggcagctcttcaatgaccgcctgtccctcgccagacaccagaatacccacactggctga

SEQ ID NO: 10 - protein, 1464 aa, Homo sapiens
MYQPEDDNNSDVTSDDDMTRNRRESSPPHSVHSFSGDRDWDRRGRSRDMEPRDRWSHTRNPRSRMPPRDLSLPVVAKTSFEMDREDDRDSRAYESRSQDAESYQNVVDLAEDRKPHNTIQDNMENYRKLLSLGVQLAEDDGHSHMTQGHSSRSKRSAYPSTSRGLKTMPEAKKSTHRRGICEDESSHGVIMEKFIKDVSRSSKSGRARESSDRSQRFPRMSDDNWKDISLNKRESVIQQRVYEGNAFRGGFRFNSTLVSRKRVLERKRRYHFDTDGKGSIHDQKGCPRKKPFECGSEMRKAMSVSSLSSLSSPSFTESQPIDFGAMPYVCDECGRSFSVISEFVEHQIMHTRENLYEYGESFIHSVAVSEVQKSQVGGKRFECKDCGETFNKSAALAEHRKIHARGYLVECKNQECEEAFMPSPTFSELQKIYGKDKFYECRVCKETFLHSSALIEHQKIHFGDDKDNEREHERERERERGETFRPSPALNEFQKMYGKEKMYECKVCGETFLHSSSLKEHQKIHTRGNPFENKGKVCEETFIPGQSLKRRQKTYNKEKLCDFTDGRDAFMQSSELSEHQKIHSRKNLFEGRGYEKSVIHSGPFTESQKSHTITRPLESDEDEKAFTISSNPYENQKIPTKENVYEAKSYERSVIHSLASVEAQKSHSVAGPSKPKVMAESTIQSFDAINHQRVRAGGNTSEGREYSRSVIHSLVASKPPRSHNGNELVESNEKGESSIYISDLNDKRQKIPARENPCEGGSKNRNYEDSVIQSVFRAKPQKSVPGEGSGEFKKDGEFSVPSSNVREYQKARAKKKYIEHRSNETSVIHSLPFGEQTFRPRGMLYECQECGECFAHSSDLTEHQKIHDREKPSGSRNYEWSVIRSLAPTDPQTSYAQEQYAKEQARNKCKDFRQFFATSEDLNTNQKIYDQEKSHGEESQGENTDGEETHSEETHGQETIEDPVIQGSDMEDPQKDDPDDKIYECEDCGLGFVDLTDLTDHQKVHSRKCLVDSREYTHSVIHTHSISEYQRDYTGEQLYECPKCGESFIHSSFLFEHQRIHEQDQLYSMKGCDDGFIALLPMKPRRNRAAERNPALAGSAIRCLLCGQGFIHSSALNEHMRLHREDDLLEQSQMAEEAIIPGLALTEFQRSQTEERLFECAVCGESFVNPAELADHVTVHKNEPYEYGSSYTHTSFLTEPLKGAIPFYECKDCGKSFIHSTVLTKHKELHLEEEEEDEAAAAAAAAAQEVEANVHVPQVVLRIQGLNVEAAEPEVEAAEPEVEAAEPEVEAAEPNGEAEGPDGEAAEPIGEAGQPNGEAEQPNGDADEPDGAGIEDPEERAEEPEGKAEEPEGDADEPDGVGIEDPEEGEDQEIQVEEPYYDCHECTETFTSSTAFSEHLKTHASMIIFEPANAFGECSGYIERASTSTGGANQADEKYFKCDVCGQLFNDRLSLARHQNTHTG

SEQ ID NO: 11 - DNA, 1159 bp, Homo sapiens
accaaccagaagacgacaacaacagtgacgtgaccagtgacgacgacatgacccggaacagaagagagtcctcaccacctcactcagtccattctttcagtggtgaccgggactgggaccggaggggcagaagcagagacatggagccacgagaccgctggtcccacaccaggaacccaagaagcaggatgcctccgcgggatctttcccttcctgtggtggcgaaaacaagctttgaaatggacagagaggacgacagggactccagggcttatgagtcccgatctcaggatgctgaatcataccaaaatgtggtggacctcgctgaggacaggaaacctcacaacacaatccaggacaacatggaaaactacaggaagctgctctccctcggagtgcagcttgctgaagacgatggccactcccacatgacgcagggccactcatcaagatccaagagaagtgcctacccaagcaccagtcgaggtctaaaaactatgcctgaagccaaaaaatcaacccaccggcgggggatttgtgaagatgaatcttcccacggagtgataatggaaaaattcatcaaggatgtgtcacgcagttccaaatcgggaagagcaagggagtcaagcgaccggtcacagagattccccagaatgtcagatgataactggaaggacatttcattgaacaagagggagtcagtgatccagcagcgggtttatgaagggaatgcatttaggggaggctttaggtttaattcaacccttgtttccagaaagagagttcttgaaagaaagaggcgctatcattttgacacagatgggaagggctcgattcacgatcaaaaaggctgtcccaggaagaagccctttgaatgtggtagtgagatgagaaaagccatgagcgtgagcagcctgagcagcctcagctccccctcctttaccgagtcacagccaattgattttggggcaatgccatatgtatgtgatgagtgtgggaggtcgttcagtgtcatctcagaatttgttgagcaccagatcatgcatactagagagaacctctatgagtatggtgagtcctttatccacagtgtggctgtcagtgaagttcagaaaagtcaggttggagggaaacgttttgaatgtaaggactgtggaga

SEQ ID NO: 12 - protein, 388 aa, Homo sapiens
QPEDDNNSDVTSDDDMTRNRRESSPPHSVHSFSGDRDWDRRGRSRDMEPRDRWSHTRNPRSRMPPRDLSLPVVAKTSFEMDREDDRDSRAYESRSQDAESYQNVVDLAEDRKPHNTIQDNMENYRKLLSLGVQLAEDDGHSHMTQGHSSRSKRSAYPSTSRGLKTMPEAKKSTHRRGICEDESSHGVIMEKFIKDVSRSSKSGRARESSDRSQRFPRMSDDNWKDISLNKRESVIQQRVYEGNAFRGGFRFNSTLVSRKRVLERKRRYHFDTDGKGSIHDQKGCPRKKPFECGSEMRKAMSVSSLSSLSSPSFTESQPIDFGAMPYVCDECGRSFSVISEFVEHQIMHTRENLYEYGESFIHSVAVSEVQKSQVGGKRFECKDCGENS

SEQ ID NO: 13 - DNA, 93 bp, Homo sapiens
gagtgcagcttgctgaagacgatggccactcccacatgacgcagggccactcatcaagatccaagagaagtgcctacccaagcaccagtcgag

SEQ ID NO: 14 - protein, 30 aa, Homo sapiens
VQLAEDDGHSHMTQGHSSRSKRSAYPSTSR

SEQ ID NO: 15 - DNA, 1173 bp, Artificial Sequence
  Description of Artificial Sequence cDNA sequence as a result of cDNA cloning, including the EcoRI linker sequence
ggaattctctccacagtccttacattcaaaacgtttccctccaacctgacttttctgaacttcactgacagccacactgtggataaaggactcaccatactcatagaggttctctctagtatgcatgatctggtgctcaacaaattctgagatgacactgaacgacctcccacactcatcacatacatatggcattgccccaaaatcaattggctgtgactcggtaaaggagggggagctgaggctgctcaggctgctcacgctcatggcttttctcatctcactaccacattcaaagggcttcttcctgggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccaccacattttggtatgattcagcatcctgagatcgggactcataagccctggagtccctgtcgtcctctctgtccatttcaaagcttgttttcgccaccacaggaagggaaagatcccgcggaggcatcctgcttcttgggttcctggtgtgggaccagcggtctcgtggctccatgtctctgcttctgcccctccggtcccagtcccggtcaccactgaaagaatggactgagtgaggtggtgaggactctcttctgttccgggtcatgtcgtcgtcactggtcacgtcactgttgttgtcgtcttctggttggtgaattcg

SEQ ID NO: 16 - DNA, 830 bp, Artificial Sequence
  Description of Artificial Sequence cDNA clone, including EcoRI linker sequence
ggaattctctccacagtccttacattcaaaacgtttccctccaacctgacttttctgaacttcactgacagccacactgtggataaaggactcaccatactcatagaggttctctctagtatgcatgatctggtgctcaacaaattctgagatgacactgaacgacctcccacactcatcacatacatatggcattgccccaaaatcaattggctgtgactcggtaaaggagggggagctgaggctgctcaggctgctcacgctcatggcttttctcatctcactaccacattcaaagggcttcttcctgggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtt

SEQ ID NO: 17 - DNA, 851 bp, Artificial Sequence
  Description of Artificial Sequence cDNA clone, including EcoRI linker sequence
ggaattctctccacagtccttacattcaaaacgtttccctccaacctgacttttctgaacttcactgacagccacactgtggataaaggactcaccatactcatagaggttctctctagtatgcatgatctggtgctcaacaaattctgagatgacactgaacgacctcccacactcatcacatacatatggcattgccccaaaatcaattggctgtgactcggtaaaggagggggagctgaggctgctcaggctgctcacgctcatggcttttctcatctcactaccacattcaaagggcttcttcctgggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtcca

SEQ ID NO: 18 - DNA, 853 bp, Artificial Sequence
  Description of Artificial Sequence cDNA clone, including EcoRI linker sequence
ggaattctctccacagtccttacattcaaaacgtttccctccaacctgacttttctgaacttcactgacagccacactgtggataaaggactcaccatactcatagaggttctctctagtatgcatgatctggtgctcaacaaattctgagatgacactgaacgacctcccacactcatcacatacatatggcattgccccaaaatcaattggctgtgactcggtaaaggagggggagctgaggctgctcaggctgctcacgctcatggcttttctcatctcactaccacattcaaagggcttcttcctgggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccacc

SEQ ID NO: 19 - DNA, 858 bp, Artificial Sequence
  Description of Artificial Sequence cDNA clone, including EcoRI linker sequence
ggaattctctccacagtccttacattcaaaacgtttccctccaacctgacttttctgaacttcactgacagccacactgtggataaaggactcaccatactcatagaggttctctctagtatgcatgatctggtgctcaacaaattctgagatgacactgaacgacctcccacactcatcacatacatatggcattgccccaaaatcaattggctgtgactcggtaaaggagggggagctgaggctgctcaggctgctcacgctcatggcttttctcatctcactaccacattcaaagggcttcttcctgggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccaccacatt

SEQ ID NO: 20 - DNA, 863 bp, Artificial Sequence
  Description of Artificial Sequence cDNA clone, including EcoRI linker sequence
ggacagcctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccaccacattttggtatgattcagcatcctgagatcgggactcataagccctggagtccctgtcgtcctctctgtccatttcaaagcttgttttcgccaccacaggaagggaaagatcccgcggaggcatcctgcttcttgggttcctggtgtgggaccagcggtctcgtggctccatgtctctgcttctgcccctccggtcccagtcccggtcaccactgaaagaatggactgagtgaggtggtgaggactctcttctgttccgggtcatgtcgtcgtcactggtcacgtcactgttgttgtcgtcttctggttggtgaattcg

SEQ ID NO: 21 - DNA, 857 bp, Artificial Sequence
  Description of Artificial Sequence cDNA clone, including EcoRI linker sequence
cctttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccaccacattttggtatgattcagcatcctgagatcgggactcataagccctggagtccctgccgtcctctctgtccatttcaaagcttgttttcgccaccacaggaagggaaagatcccgcggaggcatcctgcttcttgggttcctggtgtgggaccagcggtctcgtggctccatgtctctgcttctgcccctccggtcccagtcccggtcaccactgaaagaatggactgagtgaggtggtgaggactctcttctgttccgggtcatgtcgtcgtcactggtcacgtcactgttgttgtcgtcttctggttggtgaattcg

SEQ ID NO: 22 - DNA, 855 bp, Artificial Sequence
  Description of Artificial Sequence cDNA clone, including EcoRI linker sequence
tttttgatcgtgaatcgagcccttcccatctgtgtcaaaatgatagcgcctctttctttcaagaactctctttctggaaacaagggttgaattaaacctaaagcctcccctaaatgcattcccttcataaacccgctgctggatcactgactccctcttgttcaatgaaatgtccttccagttatcatctgacattctggggaatctctgtgaccggtcgcttgactcccttgctcttcccgatttggaactgcgtgacacatccttgatgaatttttccattatcactccgtgggaagattcatcttcacaaatcccccgccggtgggttgattttttggcttcaggcatagtttttagacctcgactggtgcttgggtaggcacttctcttggatcttgatgagtggccctgcgtcatgtgggagtggccatcgtcttcagcaagctgcactccgagggagagcagcttcctgtagttttccatgttgtcctggattgtgttgtgaggtttcctgtcctcagcgaggtccaccacattttggtatgattcagcatcctgagatcgggactcataagccctggagtccctgtcgtcctctctgtccatttcaaagcttgttttcgccaccacaggaagggaaagatcccgcggaggcatcctgcttcttgggttcctggtgtgggaccagcggtctcgtggctccatgtctctgcttctgcccctccggtcccagtcccggtcaccactgaaagaatggactgagtgaggtggtgaggactctcttctgttccgggtcatgtcgtcgtcactggtcacgtcactgttgttgtcgtcttctggttggtgaattcg

SEQ ID NO: 23 - DNA, 32 bp, Artificial Sequence
  Description of Artificial Sequence Primer
gatcgaattcaccaaccagaagacgacaacaa

SEQ ID NO: 24 - DNA, 32 bp, Artificial Sequence
  Description of Artificial Sequence Primer
gatcgaattctctccacagtccttacattcaa

SEQ ID NO: 25 - DNA, 10 bp, Artificial Sequence
  Description of Artificial Sequence EcoRI linker sequence
gatcgaattc

(19) United States
(12) Patent Application Publication
(10) Pub. No.: US 2002/0182668 A1
(43) Pub. Date: Dec. 5, 2002

(54) Human PEG3 gene and uses thereof

(76) Inventors: Azim Surani, Cambridge (GB); Yuk Yee Szeto, Cambridge (GB)

Correspondence Address: Dean H. Nakamura, Roylance Abrams Berdo & Goodman, 1300 19th Street, N.W., Washington, DC, 20036, US

(21) Appl. No.: 09/842,777

(22) Filed: Apr. 27, 2001

(51) Int. Cl. C12Q 1/68; C07H 21/04; C12N 9/22; C12P 21/02

(52) U.S. Cl. 435/69.1; 435/6; 435/199; 536/23.2

(57) ABSTRACT

The invention provides isolated nucleic acid (SEQ ID NO:9) encoding human PEG3 (paternally expressed gene 3), and the use of the nucleic acid in diagnostic assays.

### FIELD OF THE INVENTION

The present invention relates to the human PEG3 gene and its use.

### BACKGROUND TO THE INVENTION

The human gene PEG3 (paternally expressed gene 3) is a zinc finger protein which is believed to act as a transcription factor. The murine homologue of this gene, Peg3, has been found to play a role in maternal behaviour, thermoregulation and weight regulation. Studies of mice in which the gene has been inactivated show that such mice have a tendency to become obese, and females of the species exhibit poor maternal behaviour.

Kim et al, (Genomics 64, 114-118, (2000)) describe a schematic layout of the exon structure of the human PEG3 gene. They indicate that the first seven exons of PEG3 are shared with another gene, ZIM2, whose remaining four exons are located downstream of the final two exons of PEG3. Amino acid sequences of exons 3-7 are described. Genbank accession number AC006115 provides genomic sequence of a region, which includes at least part of the PEG3 gene. The Genbank entry provides a proposed translation of a region of the gene, which, in the light of the present invention, we have determined is part of the C-terminus of exon 9 of PEG3.

### DISCLOSURE OF THE INVENTION

In order to facilitate study of PEG3, a complete cDNA clone is desirable. Prior to the present invention, the full open reading frame of PEG3 was unknown.

In a first aspect, the present invention is based on the provision of a cDNA structure which includes a complete exon 8 of PEG3, together and spliced to it at the 5′ junction with exon 7 and at the 3′ junction at least part of exon 9. Assembly of this clone with the remainder of exons 3-7 and 9 provides the full open reading frame of PEG3 for the first time.

Thus in a first aspect, the invention provides an isolated cDNA having the sequence of SEQ ID NO:1.

In another aspect, the invention provides an isolated cDNA fragment of SEQ ID NO:1, said fragment being selected from the group SEQ ID NO:2-8.

In a further aspect, the invention provides an isolated cDNA having the sequence of SEQ ID NO:9.

In another aspect, the invention provides an isolated cDNA having the sequence of SEQ ID NO:13.

The invention further provides the use of these and other nucleic acids for the expression of PEG3 protein, or portions thereof, and in assay and diagnostic methods.

### DESCRIPTION OF THE DRAWINGS

FIG. 1 shows the raw data produced from the seven sequence reactions of seven clones obtained by PCR amplification of placental cDNA using the primers HFL1 (SEQ ID NO:23) and HFL2 (SEQ ID NO:24), together with an alignment of the these clones to show how sequence SEQ ID NO:1 was arrived at. In FIG. 1, those of skill in the art will recognise that the sequence includes EcoR1 linkers.

### SUMMARY OF SEQ ID NOS.

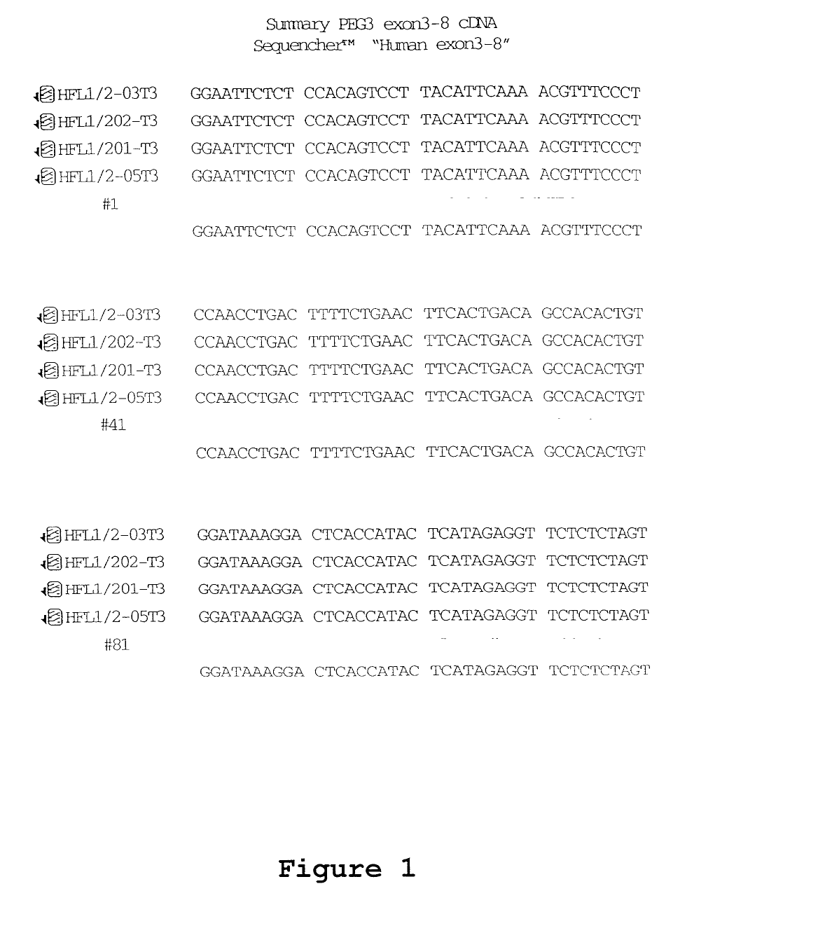

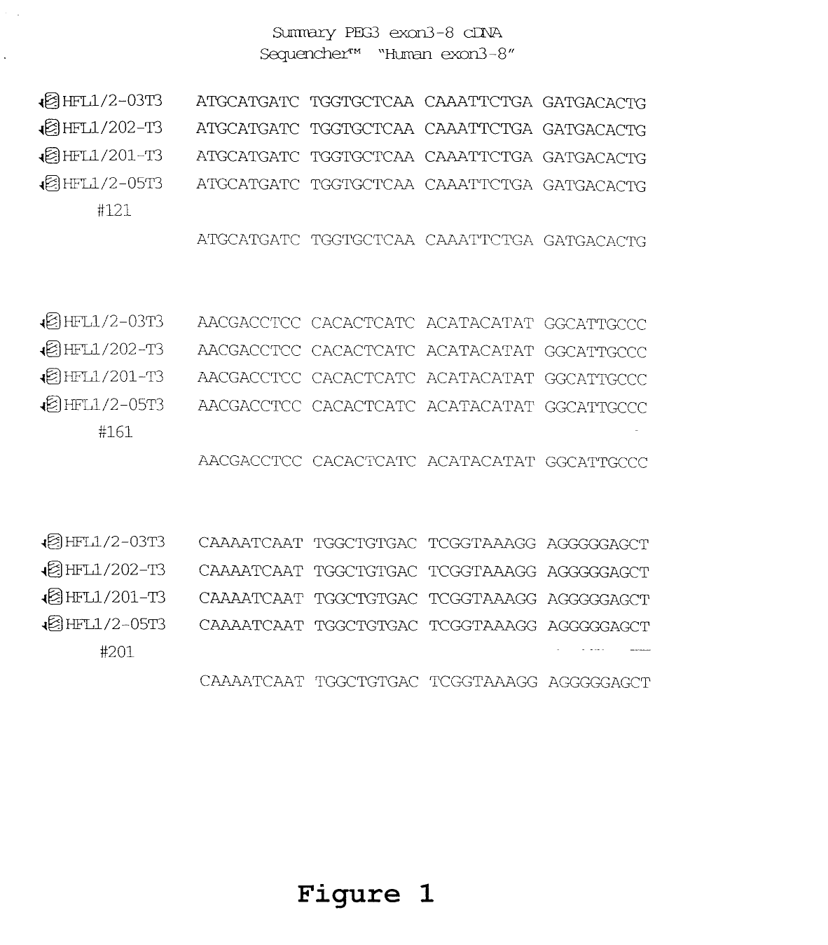

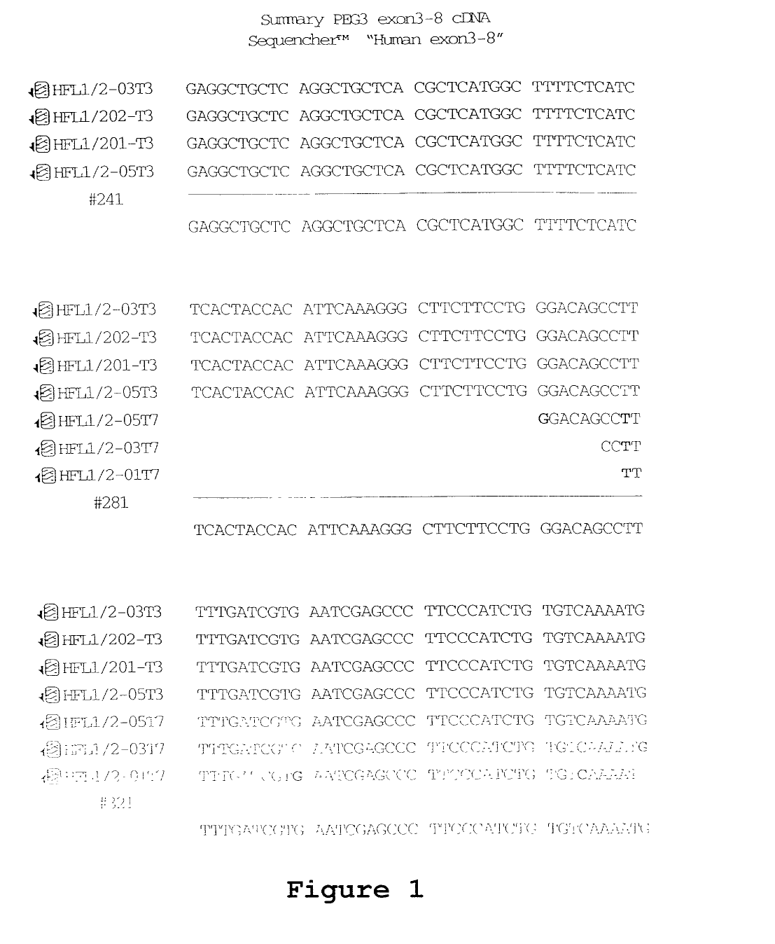

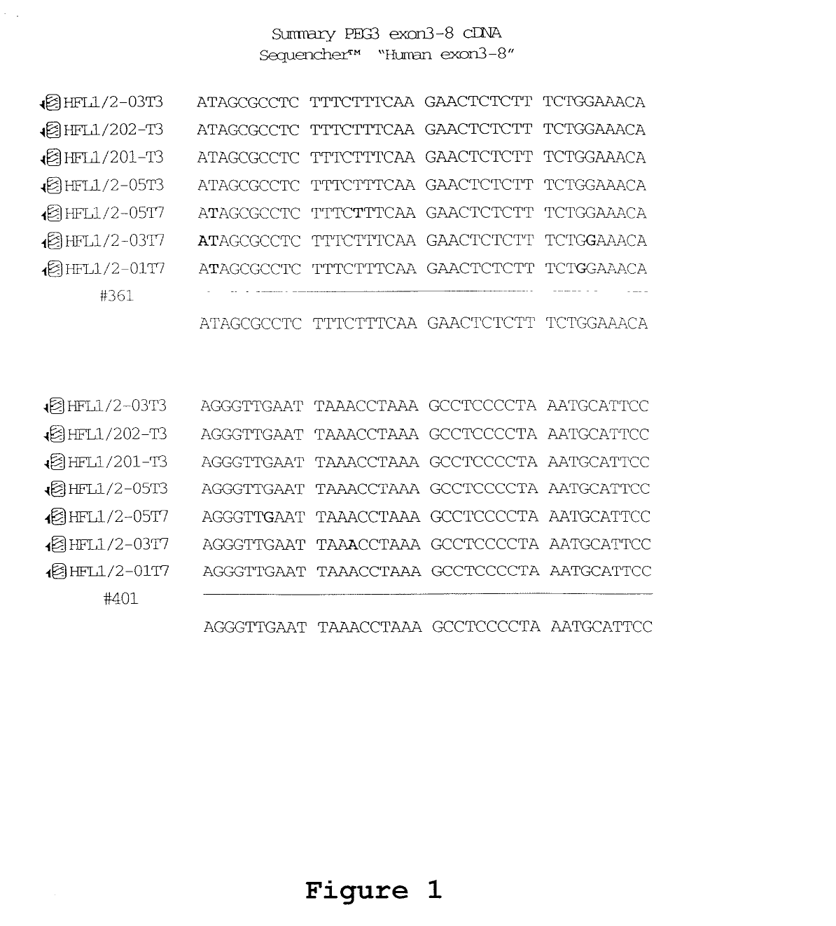

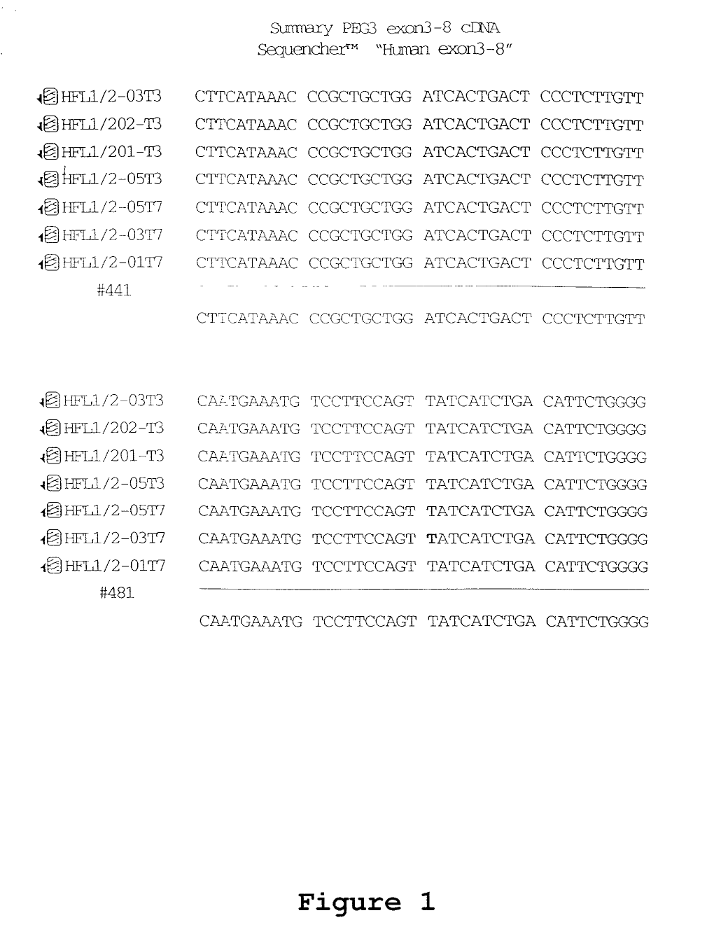

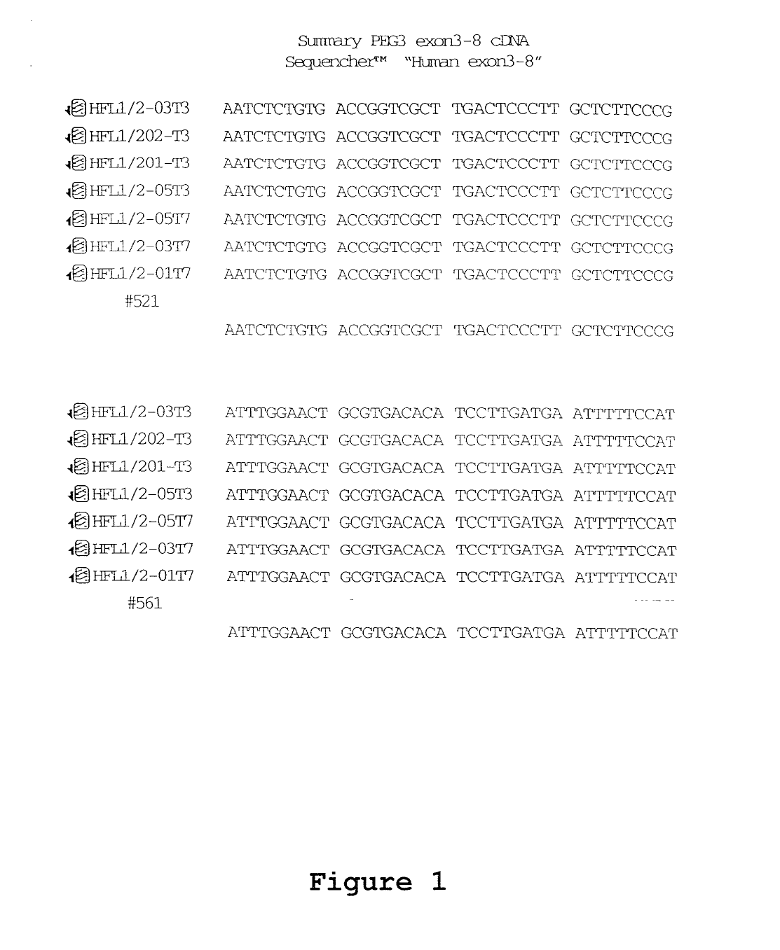

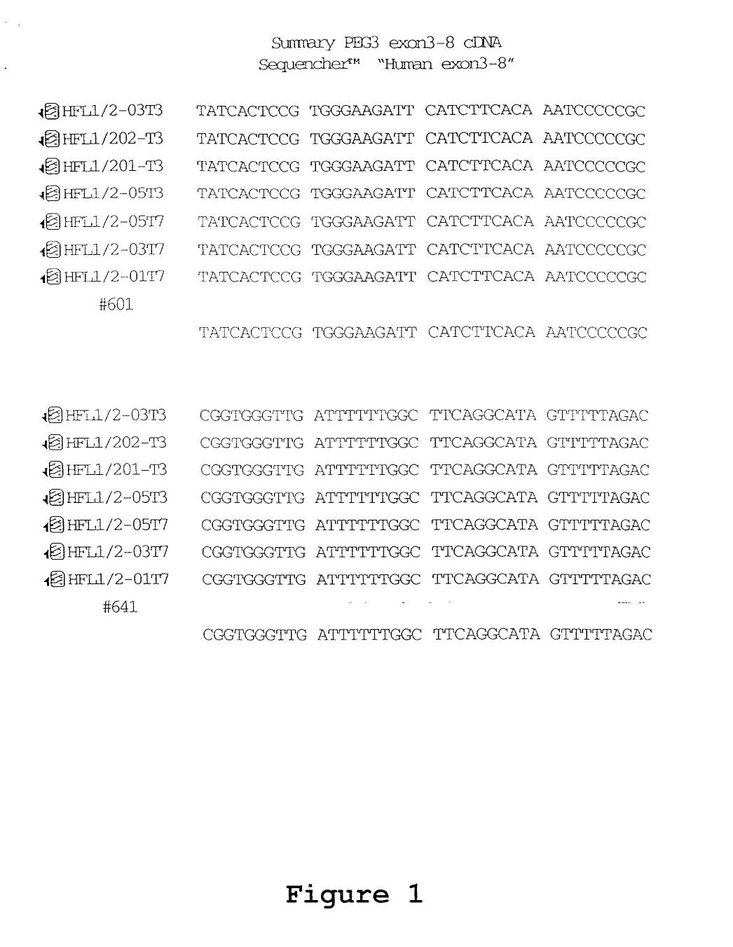

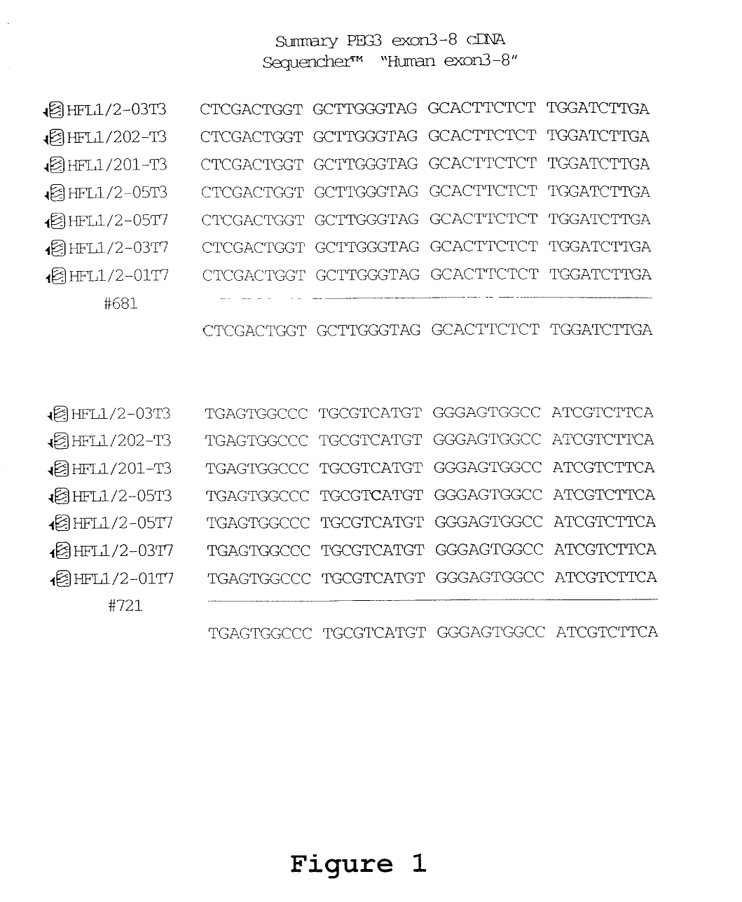

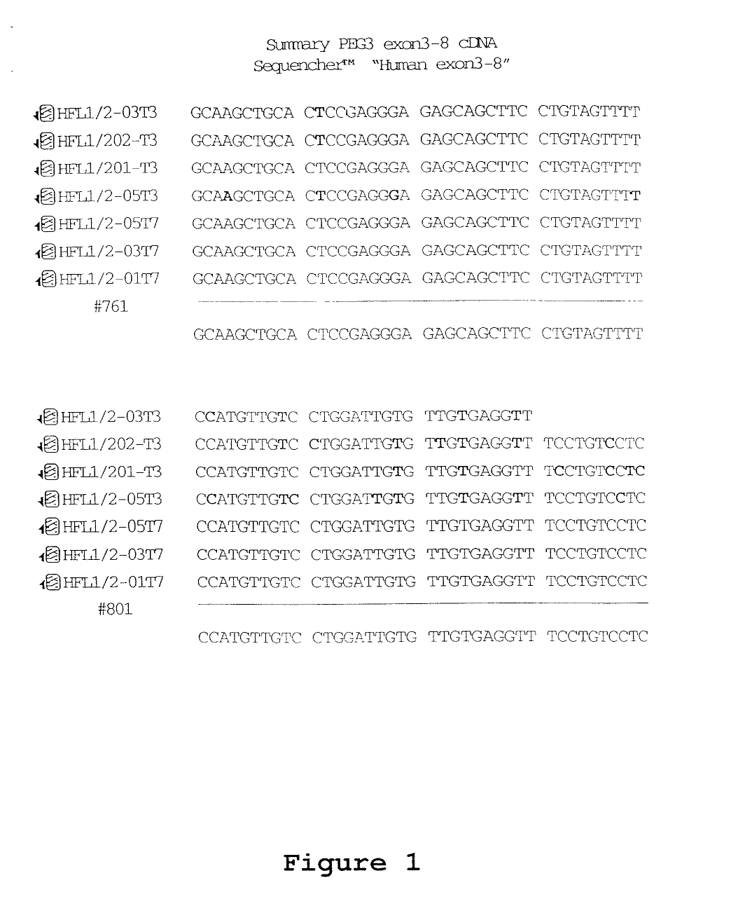

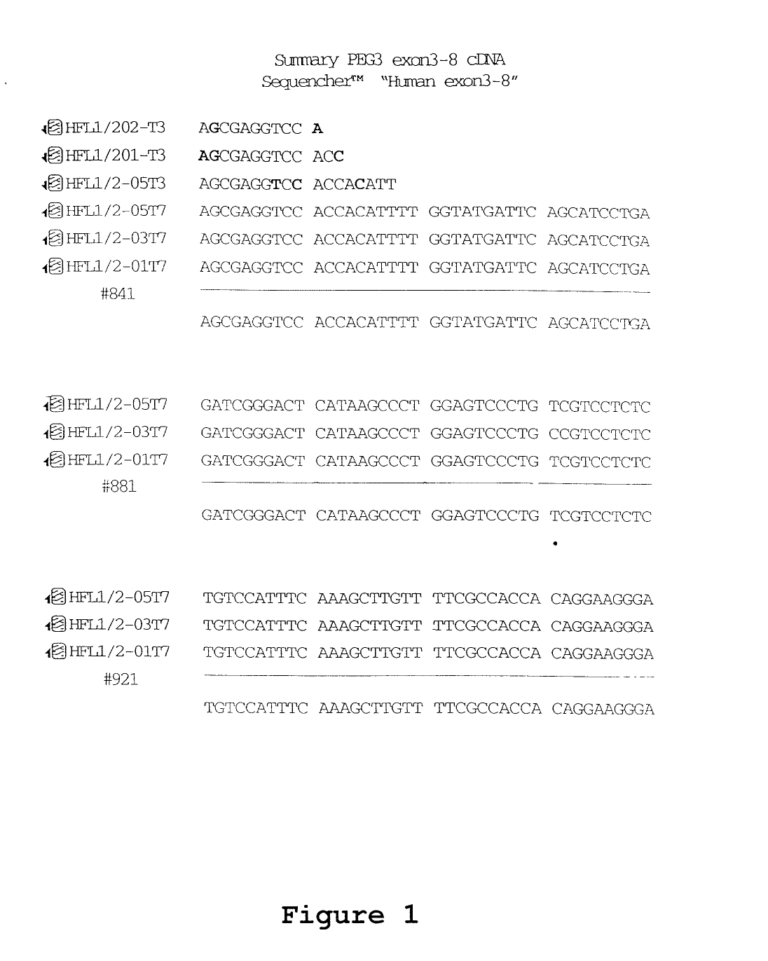

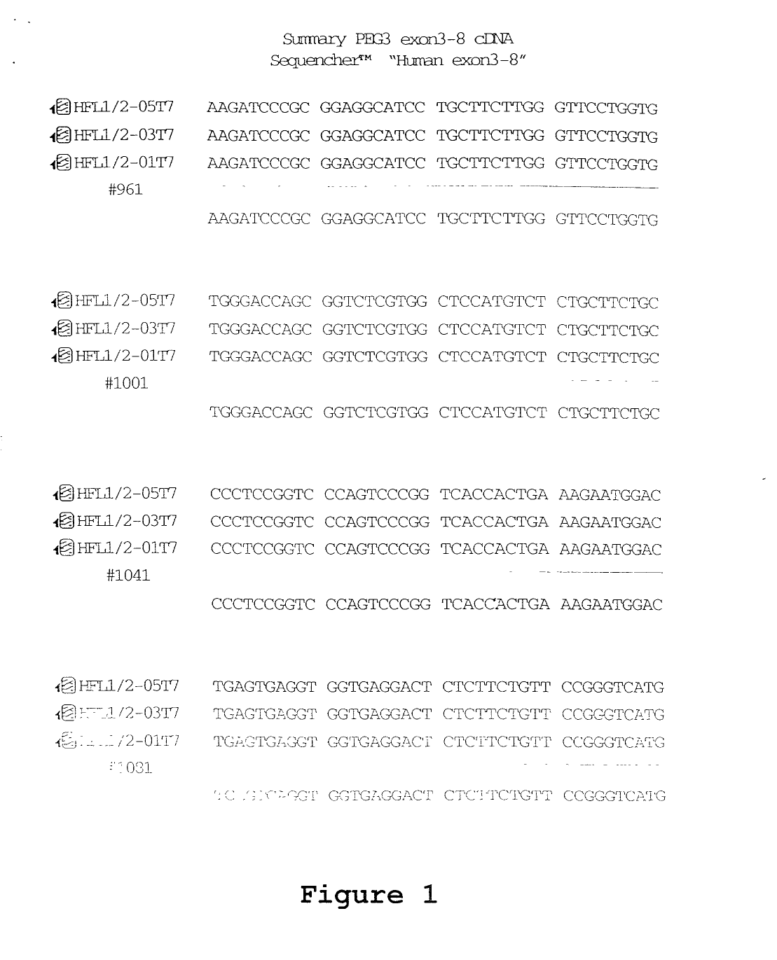

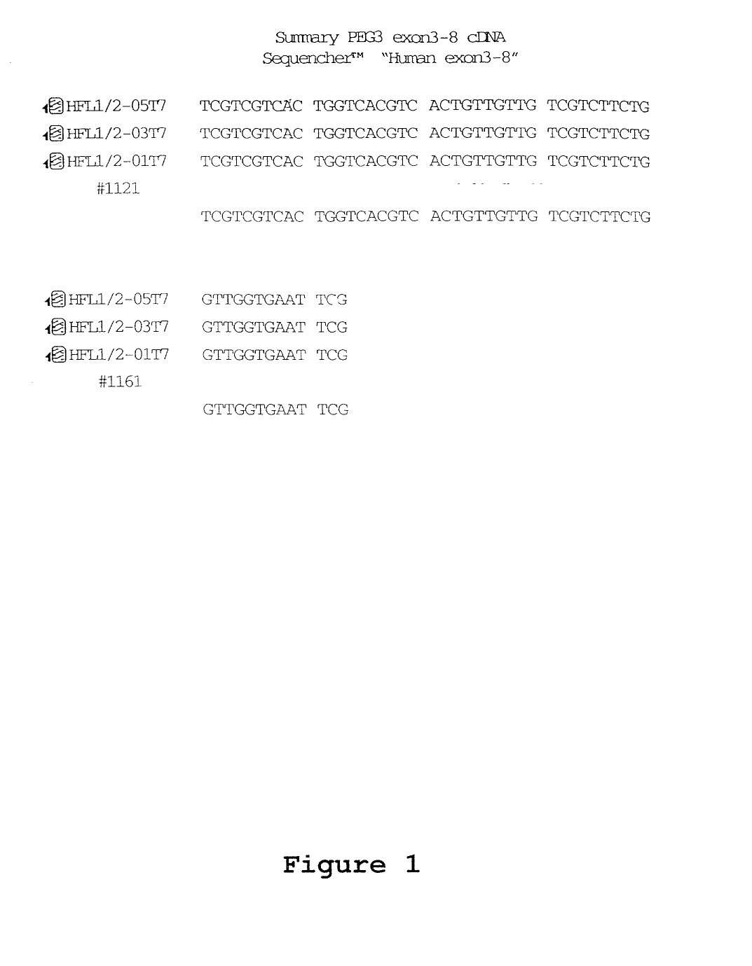

SEQ ID NO:1 is the cDNA sequence of FIG. 1, excluding the EcoR1 linker sequence, which those of skill in the art will immediately appreciate are a result of the PCR cloning process, and not of natural origin.

SEQ ID NOs:2-8 are the sequences of individual clones which were used to determine SEQ ID NO:1. As with SEQ ID NO:1, these also exclude RI linker sequences. SEQ ID NOs:6-8 are presented and listed in the opposite orientation to which they were sequenced, in order to align them with SEQ ID NOs:2-5.

SEQ ID NO:9 is the full open reading frame of PEG3 cDNA, together with 363 nucleotides of the 5′ untranslated region. The ORF is from 364 to 4755, with 4756-4758 being the stop codon TGA. Exon 8 of PEG3 (SEQ ID NO:13) is at 761-853 of SEQ ID NO:9.

SEQ ID NO:10 is the translation of the full open reading frame of SEQ ID NO:9.

SEQ ID NO:11 is the opposite (sense) strand of SEQ ID NO:1.

SEQ ID NO:12 is the translation of SEQ ID NO:11.

SEQ ID NO:13 is exon 8 of PEG3.

SEQ ID NO:14 is the translation of SEQ ID NO:13.

SEQ ID NOs:15-22 are the sequences shown in FIG. 1, i.e. including R1 linker sequences.

SEQ ID NO:23 is the 5′ primer (HFL1) used to clone SEQ ID NO:1.

SEQ ID NO:24 is the 3′ primer (HFL2) used to clone SEQ ID NO:1.

### DETAILED DESCRIPTION OF THE INVENTION.

In addition to SEQ ID NO:1, the invention provides further novel DNA and RNA sequences. These are collectively referred to as “nucleic acid(s)” or “polynucleotide(s)” of the invention. These terms are used interchangeably and are equivalent. Nucleic acids of the invention may be single or double stranded.

Thus the invention provides fragments of SEQ ID NO:1, particularly fragments corresponding to the complement of 761-853 of SEQ ID NO:9 and subportions thereof. Such subportions may be at least 15, preferably at least 20, preferably at least 25, more preferably at least 50 nucleotides in length. Such fragments may be cloned into suitable vectors including expression vectors or may be isolated as double or single strand nucleic acids and used as probes. Subportions in the range of 15 to 30, such as 15 to 25 nucleotides in length may be provided in single stranded form and are useful as probes and primers, particularly PCR primers. PCR primers may additionally comprise, usually at their 5′ end, a linker sequence providing a restriction endonuclease recognition site, facilitating cloning of a PCR amplified product.

SEQ ID NO:1 of the present invention can be obtained by using the primers HFL1 (SEQ ID NO:23) and HFL2 (SEQ ID NO:24) as PCR primers on a sample of mRNA or cDNA from a suitable human cell source. Placental mRNA or cDNA is particularly useful, though other cell sources include brain, ovary, testis, heart and pancreas. mRNA may be isolated from placental material. cDNA libraries from cell sources including placenta are available commercially, for example from CLONTECH.

PCR may be performed under standard conditions, which are well known in the art.

Nucleic acid of the invention may be cloned into a suitable vector, preferably a vector with a unique EcoR1 site. pUC vectors and derivatives thereof are particularly suitable for this purpose, such as the pBS SKII(+) vector from Stratagene.

The nucleic acids of the invention may also be cloned into expression vectors in an orientation so as to provide for expression of the cDNA encoding all or part of PEG3 protein.

For expression of SEQ ID NO:1 or other nucleic acids of the invention, the nucleic acid sequence will be operably linked in-frame to a promoter to provide for transcription of the sequence. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage phagemid or baculoviral, cosmids, YACs, BACs, or PACs as appropriate. Vectors include gene therapy vectors, for example vectors based on adenovirus, adeno-associated virus, retrovirus (such as HIV or MLV) or alpha virus vectors.

The vectors may be provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used in vitro, for example for the production of RNA or used to transfect or transform a host cell. The vector may also be adapted to be used in vivo, for example in methods of gene therapy. Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous olypeptide include Chinese hamster ovary cells, HeLa cells, baby hamster kidney cells, COS cells and many others.

Promoters and other expression regulation signals may be selected to be compatible with the host cell for which the expression vector is designed. For example, yeast promoters include S. cerevisiae GAL4 and ADH promoters, S. pombe nmtl and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

The vectors may include other sequences such as promoters or enhancers to drive the expression of the inserted nucleic acid, nucleic acid sequences so that the polypeptide is produced as a fusion and/or nucleic acid encoding secretion signals so that the polypeptide produced in the host cell is secreted from the cell.

Vectors may also include sequences which encode reporter genes such as β-galactosidase, which genes are fused in-frame to the nucleic acid of the invention.

Vectors for production of polypeptides of the invention of for use in gene therapy include vectors which carry a mini-gene sequence of the invention.

For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Vectors may be transformed into a suitable host cell as described above to provide for expression of a polypeptide of the invention. Thus, in a further aspect the invention provides a process for preparing polypeptides according to the invention which comprises cultivating a host cell transformed or transfected with an expression vector as described above under conditions to provide for expression by the vector of a coding sequence encoding the polypeptides, and recovering the expressed polypeptides. Polypeptides may also be expressed in in vitro systems, such as reticulocyte lysate.

Polynucleotides according to the invention may also be inserted into the vectors described above in an antisense orientation in order to provide for the production of antisense RNA or ribozymes.

A still further aspect of the present invention provides a method which includes introducing the nucleic acid into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. As an alternative, direct injection of the nucleic acid could be employed.

The introduction may be followed by causing or allowing expression from the nucleic acid, e.g. by culturing host cells (which may include cells actually transformed although more likely the cells will be descendants of the transformed cells) under conditions for expression of the gene, so that the encoded polypeptide is produced. If the polypeptide is expressed coupled to an appropriate signal leader peptide it may be secreted from the cell into the culture medium. Following production by expression, a polypeptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers (e.g. see below).

A further aspect of the present invention provides a host cell containing nucleic acid as disclosed herein. The cells will be chosen to be compatible with the said vector and may for example be bacterial, yeast, insect or mammalian. The nucleic acid of the invention may be integrated into the genome (e.g. chromosome) of the host cell. Integration may be promoted by inclusion of sequences which promote recombination with the genome, in accordance with standard techniques. The nucleic acid may be on an extra-chromosomal vector within the cell.

Cell lines may be used in a method for assaying a putative modulator of PEG3 (e.g. a modulator of body weight, thermoregulation, etc), which method comprises bringing a putative modulator into contact with the cell line and determining the effect of said modulator on transcription, translation or mRNA or protein turnover of a nucleic acid or expressed polypeptide of the invention. Modulators may also be examined for their effects on translocation and DNA binding of PEG3. Modulators which have an effect on this may be used as modulators of PEG3.

Nucleic acids of the invention, particularly when in the form of a recombinant vector, may be used in methods of gene therapy. A construct capable of expressing a nucleic acid of the invention may be introduced into cells of a recipient by any suitable means, such that a PEG3 polypeptide of the invention is expressed in the cells.

The construct may be introduced in the form of naked DNA, which is taken up by some cells of animal subjects, including muscle cells of mammalians. In this aspect of the invention the construct will generally be carried by a pharmaceutically acceptable carrier alone. The construct may also be formulated in a liposome particle.

Such methods of gene therapy further include the use of recombinant viral vectors such as adenoviral or retroviral vectors which comprise a construct capable of expressing a polypeptide of the invention. Such viral vectors may be delivered to the body in the form of packaged viral particles.

Constructs of the invention, however formulated and delivered, may be for use in treating conditions brought about by a defect in the PEG3 locus. The construct will comprise nucleic acid encoding PEG3 linked to a promoter capable of expressing the gene in the target cells. The constructs may be introduced into cells of a human or non-human mammalian recipient either in situ or ex-vivo and reimplanted into the body. Where delivered in situ, this may be by for example injection into target tissue(s) or in the case of liposomes, inhalation.

Gene therapy methods are widely documented in the art and may be adapted for use in the expression of a polypeptide of the invention. See for example WO95/14091 and Walther, Molecular Biotechnology, 6(3): 267-286, (1996) and Blomer, Human Molecular Genetics, Vol.5: 1397-1404, (1996), the disclosures of which are incorporated herein by reference.

Human PEG3 nucleic acid sequences of the invention may be used in methods of gene therapy, to increase or, in the form of antisense nucleic acid, decrease, the expression of PEG3 in cells of a human patient. Such therapy may be used to modify or control body weight and/or thermoregulation in the patient. Nucleic acid may be delivered to cells such as adipose tissue in the patient where expression may be particularly desired.

DNA sequences of the invention including fragments of SEQ ID NO:1 in the form of probes may be used to detect the presence or absence of PEG3 DNA or mRNA in a sample. Detection may be qualitative or quantitative. For example, levels of PEG3 expression may be associated with a number of conditions including thermoregulation and obesity. Levels of PEG3 expression may be determined in order to assess expression levels of the gene in tissues as a means of diagnosis or prognosis of such conditions and to monitor changes in expression level, for example in response to a course of treatment.

Such detection means may include a process which comprises providing a sample of DNA or RNA from an individual, bringing a nucleic acid of the invention into contact with the sample under conditions which allow the DNA of the invention to hybridise to a complementary sequence in the sample, and detecting whether or not such hybridisation has occurred. To facilitate detection, the DNA of the invention may be labelled with a detectable label such as a radiolabel, a fluorescent label or an amplifiable label such as biotin. Alternatively, detection may utilise a short sequence of the invention as one member of a PCR primer pair which may be used in conjunction with a second primer sequence which targets a complementary strand of the PEG gene such that a PCR reaction produces a detectable amplified product.

The provision of the sequence of SEQ ID NO:1 allows for the first time the assembly of a full open reading frame of the human PEG3 gene. Thus, the sequence of the invention may be linked at the 5′ terminus to the four additional nucleotides required to provide the ATG initiation codon and the first nucleotide of codon 2, and, at the 3′ end to the remainder of exon 9. This provides the full open reading frame shown herein as SEQ ID NO:9. The sequences from the prior art may be obtained in any suitable form, for example as genomic fragments or cDNA sequences, and assembled using recombinant DNA techniques known as such in the art. For example, see Sambrook et al, 1987, Cold Spring Harbour which provides examples of suitable methods and materials for recombinant DNA manipulation.

The assembled clone may be inserted into an expression vector including expression vectors of the type above and used for the expression and recovery in isolated form of human PEG3 protein.

Thus in a further aspect, the invention provides an isolated polypeptide comprising SEQ ID NO:10. The invention further provides isolated polypeptides which are fragments of SEQ ID NO:10, said fragments comprising SEQ ID NO:14 or a fragment thereof of at least 15 amino acids of SEQ ID NO:14, such as at least 20 or at least 25 amino acids of SEQ ID NO:14.

Isolated polypeptides of the invention will be those as defined above in isolated form, free or substantially free of material with which it is naturally associated such as other polypeptides with which it is found in the cell. The polypeptides may of course be formulated with diluents or adjuvants and still for practical purposes be isolated - for example the polypeptides will normally be mixed with gelatin or other carriers if used to coat microtitre plates for use in immunoassays. The polypeptides may be glycosylated, either naturally or by systems of heterologous eukaryotic cells, or they may be (for example if produced by expression in a prokaryotic cell) unglycosylated. Polypeptides may be phosphorylated and/or acetylated.

A polypeptide of the invention may also be in a substantially purified form, in which case it will generally comprise the polypeptide in a preparation in which more than 90%, e.g. 95%, 98% or 99% of the polypeptide in the preparation is a polypeptide of the invention.

Polypeptides of the invention may be modified for example by the addition of histidine residues to assist their purification or by the addition of a signal sequence to promote their secretion from a cell.

A polypeptide according to the present invention may be isolated and/or purified (e.g. using an antibody) for instance after production by expression from encoding nucleic acid. Polypeptides according to the present invention may also be generated wholly or partly by chemical synthesis, for example in a step-wise manner. The isolated and/or purified polypeptide may be used in formulation of a composition, which may include at least one additional component, for example a pharmaceutical composition including a pharmaceutically acceptable excipient, vehicle or carrier. A composition including a polypeptide according to the invention may be used in prophylactic and/or therapeutic treatment.

Thus the invention provides a composition of the invention for use in a method of treatment of body weight, thermoregulation or behaviour, which method comprises administering to a patient in need of treatment an effective amount of a polypeptide or composition thereof of the invention.

A polypeptide according to the present invention may be used as an immunogen or otherwise in obtaining specific antibodies. Antibodies are useful in purification and other manipulation of polypeptides and peptides, diagnostic screening and therapeutic contexts. This is discussed further below.

A polypeptide according to the present invention may be used in screening for molecules which affect or modulate its activity or function. Such molecules may be useful in a therapeutic (possibly including prophylactic) context. Such screening may take the form of direct in vitro screening for modulators which bind to a polypeptide of the invention. A polypeptide of the invention may be labelled with a revealing label. The revealing label may be any suitable label which allows the polypeptide to be detected. Suitable labels include radioisotopes, e.g. ¹²⁵I, enzymes, antibodies, polynucleotides and linkers such as biotin. Labelled polypeptides of the invention may be used in diagnostic procedures such as immunoassays in order to determine the amount of a polypeptide of the invention in a sample. Polypeptides or labelled polypeptides of the invention may also be used in serological or cell mediated immune assays for the detection of immune reactivity to said polypeptides in animals and humans using standard protocols.

A polypeptide or labelled polypeptide of the invention or fragment thereof may also be fixed to a solid phase, for example the surface of an immunoassay well or dipstick.

Such labelled and/or immobilized polypeptides may be packaged into kits in a suitable container along with suitable reagents, controls, instructions and the like.

Such polypeptides and kits may be used in methods of detection of antibodies to such polypeptides present in a sample or active portions or fragments thereof by immunoassay.

Immunoassay methods are well known in the art and will generally comprise:

(a) providing a polypeptide comprising an epitope bindable by an antibody against PEG3;

(b) incubating a biological sample with said polypeptide under conditions which allow for the formation of an antibody-antigen complex; and

(c) determining whether antibody-antigen complex comprising said polypeptide is formed.

Similarly, antibodies of the invention (as defined herein below) which are capable of binding diagnostically significant epitopes of PEG3 may likewise be packaged in a kit with controls, instructions, and the like. Immunoassay methods using such antibodies will generally comprise:

(a) providing an antibody of the invention capable of binding an epitope of PEG3;

(b) incubating a biological sample with said antibody under conditions which allow for the formation of an antibody-antigen complex; and

(c) determining whether antibody-antigen complex comprising PEG3 is formed.

The identification of the polypeptide expressed by the PEG3 gene enables assays to be developed to identify further cellular proteins with which the polypeptide is associated. For example, polypeptides of the present invention form part of regulatory pathways in which their function is to interact with other factors which in turn promote or maintain essential cellular functions. The polypeptides of the present invention may be used in two-hybrid assays to determine cellular factors with which they become associated.

Two-hybrid assays may be in accordance with those disclosed by Fields and Song, 1989, Nature 340; 245-246. In such an assay the DNA binding domain (DBD) and the transcriptional activation domain (TAD) of the yeast GAL4 transcription factor are fused to the first and second molecules respectively whose interaction is to be investigated. A functional GAL4 transcription factor is restored only when two molecules of interest interact. Thus, interaction of the molecules may be measured by the use of a reporter gene operably linked to a GAL4 DNA binding site which is capable of activating transcription of said reporter gene. Other transcriptional activator domains may be used in place of the GAL4 TAD, for example the viral VP16 activation domain. In general, fusion proteins comprising DNA binding domains and activation domains may be made.

The use of a two-hybrid approach allows isolation of the gene (or at least a portion thereof which may be used to clone the whole gene) encoding the protein which interacts with a polypeptide of the invention.

An alternative approach is an immunoprecipitation. Antibodies against a polypeptide of the invention may be made, and used to immunoprecipitate this protein from cells in which it is produced, under conditions wherein a protein associated with PEG3 co-precipitates. The protein may be analysed by traditional protein chemistry, and primary sequence used to design probes to clone it. Alternatively, the primary sequence data may be compared against EST databases for candidate genes.

Isolated sequences of nucleic acids obtainable in this way form a further aspect of the invention.

In the present case polypeptides of the invention may be expressed as fusion proteins with an appropriate domain and candidate second polypeptides with which those of the invention might associate can be produced as fusion proteins with an appropriate corresponding domain. Alternatively libraries such as phage display libraries of such fusion proteins may be screened with a fusion polypeptide of the invention.

The PEG3 protein and fragments thereof may be used to obtain antibodies against PEG3. In particular, antibodies raised against the protein sequence encoded by exon 8 will be useful in distinguishing between PEG3 and the protein ZIM2 because the first seven exons of both proteins are shared. In addition, antibodies may be specific for epitopes encoded by exon 8 of human PEG3 in that they will be able to distinguish between them and epitopes of PEG3 homologues from other species, particularly murine.

Antibodies according to the invention may be useful in diagnostic and prognostic methods as discussed herein. Antibodies are also useful in purifying the polypeptide or polypeptides to which they bind, e.g. following production by recombinant expression from encoding nucleic acid.

Preferred antibodies according to the invention are isolated, in the sense of being free from contaminants such as antibodies able to bind other polypeptides and/or free of serum components. Monoclonal antibodies are preferred for some purposes, though polyclonal antibodies are within the scope of the present invention.

Antibodies may be obtained using techniques which are standard in the art. Methods of producing antibodies include immunising a mammal (e.g. mouse, rat, rabbit) with a polypeptide of the invention. Antibodies may be obtained from immunised animals using any of a variety of techniques known in the art, and screened, preferably using binding of antibody to antigen of interest. For instance, Western blotting techniques or immunoprecipitation may be used (Armitage et al, Nature, 357:80-82, 1992).

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Antibodies according to the present invention may be modified in a number of ways. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope.

Example antibody fragments, capable of binding an antigen or other binding partner include Fab fragments and Fv fragments, including single chain Fv fragments.

Humanized antibodies in which CDRs from a non-human source are grafted onto human framework regions, typically with the alteration of some of the framework amino acid residues, to provide antibodies which are less immunogenic than the parent non-human antibodies, are also included within the present invention.

Hybridomas and other host cells capable of producing antibody with desired binding characteristics are within the scope of the present invention. The invention also provides methods of production of the antibodies including growing a cell capable of producing the antibody under conditions in which the antibody is produced, and preferably secreted.

The reactivities of antibodies in a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. One favoured mode is by covalent linkage of each antibody with an individual fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. Biotin/avidin or biotin/streptavidin and alkaline phosphatase detection systems may be employed.

The mode of determining binding is not a feature of the present invention and those skilled in the art are able to choose a suitable mode according to their preference and general knowledge.

Antibodies according to the present invention may be used in screening for the presence of PEG3 for example in a test sample containing cells or cell lysate as discussed, and may be used in purifying and/or isolating a polypeptide according to the present invention, for instance following production of the polypeptide by expression from encoding nucleic acid therefore. Antibodies may modulate the activity of the polypeptide to which they bind and so, if that polypeptide has a deleterious effect in an individual, may be useful in a therapeutic context (which may include prophylaxis). An antibody may be provided in a kit, which may include instructions for use of the antibody, e.g. in determining the presence of a particular substance in a test sample. One or more other reagents may be included, such as labelling molecules, buffer solutions, elutants and so on. Reagents may be provided within containers which protect them from the external environment, such as a sealed vial.

Thus in one aspect the invention provides a method of detecting human PEG3 protein in a sample which method comprises providing a sample from a human subject, incubating the sample with an antibody against PEG3, and detecting whether or not said antibody has bound.

The provision of the human PEG3 gene allows the search for other genes involved in metabolic regulation, and thus the invention also provides a method for screening for genes associated with the regulation of body weight or temperature, which method comprises providing a PEG3 polypeptide of the present invention, bringing said polypeptide into contact with other cellular proteins, and determining to which cellular proteins the polypeptide is able to bind. Such genes may be isolated and this forms a further aspect of the invention.

The invention also allows the determination of a PEG3 genotype in humans with a phenotypic disorder associated with obesity or thermoregulation, by a method forming a further aspect of the invention. This method comprises:

providing nucleic acid from a group of obese patients and a control group of non-obese patients;

analysing said nucleic acid with a nucleic acid of the present invention; and

determining one or more features of said nucleic acid associated with the obese phenotype.

An analogous method may be performed at the protein level, using antibodies of the present invention to determine epitopes of PEG3 which differ between groups, either in the relative strength of binding or in their presence or absence.

The differences in protein epitopes or genotype determined by such screening may be utilized to provide a method of testing an individual's susceptibility to obesity or a thermoregulatory disorder which comprises analysing the nucleic acid of said individual for one or more features of the PEG3 gene associated obesity or thermoregulatory disorders.

In a further embodiment of the invention, there is provided a non-human animal which expresses the human nucleic acid of the present invention. This may be in addition to, or in place of an endogenous PEG3 gene. The provision of animals with copies of the human PEG3 gene may be achieved using the techniques known as such in the art for targeted homologous replacement of genes.

Generally, a construct containing nucleic acid of the invention will be introduced into a targeting vector which has additionally a sequence of homology with the animals endogenous PEG3 gene, and this is introduced into ES cells by electroporation, lipofection or microinjection. In a few ES cells, the targeting vector pairs with the homologous chromosomal DNA sequence and transfers the nucleic acid carried by the vector into the genome by homologous recombination. The vector is configured so that the recombination results in a knock out of the endogenous gene and the provision of a nucleic acid of the invention capable of being expressed. It may be expressed from a heterologous promoter, or from the animal's endogenous PEG3 promoter. Screening or enrichment procedures are used to identify the transfected cells, and a transfected cell is cloned and maintained as a pure population.

Next, the altered ES cells are injected into the blastocyst of a preimplantation mouse embryo or alternatively an aggregation chimera is prepared in which the ES cells are placed between two blastocysts which, with the ES cells, merge to form a single chimeric blastocyst. The chimeric blastocyst is surgically transferred into the uterus of a foster mother where the development is allowed to progress to term. The resulting animal will be a chimera of normal and donor cells. Typically the donor cells will be from a animal with a clearly distinguishable phenotype such as skin colour, so that the chimeric progeny is easily identified. The progeny is then bred and its descendants cross-bred, giving rise to heterozygotes and homozygotes for the targeted mutation. The production of transgenic animals is described further by Capecchi, M, R., 1989, Science 244; 1288-1292; Valancius and Smithies, 1991, Mol. Cell. Biol. 11; 1402-1408; and Hasty et al, 1991, Nature 350; 243-246, the disclosures of which are incorporated herein by reference.

In another aspect, animals are provided in which the human PEG3 nucleic acid of the invention is expressed at an ectopic location. This means that the gene is expressed in a location or at a time during development which does not occur in a wild-type animal. For example, the gene may be linked to a developmentally regulated promoter such as Wnt-1 and others (Echeland, Y. Et al., Development 120, 2213-2224, 1998; Rinkenberger, J. C. et al., Dev. Genet. 21, 6-10, 1997, or a tissue specific promoter such as HoxB (Machonochie, M. K. et al, Genes & Dev 11, 1885-1895, 1997).

Animals of this aspect of the invention may also be used as models in the development of assays for modulators of obesity, temperature regulation or behavioural disorders.

Non-human animals of the invention may be homozygous or heterozygous for the nucleic acid of the invention. Mammalian animals include non-human primates, rodents, rabbits, sheep, cattle, goats, pigs. Rodents include mice, rats, and guinea pigs. Amphibians include frogs. Fish such as zebra fish, may also be used.

Transgenic non-human mammals of the invention may be used for experimental purposes in studying obesity, thermoregulation or behavioural disorders, and in the development of therapies designed to alleviate the symptoms or progression of such conditions cause by a defect in the PEG3 gene. By “experimental” it is meant permissible for use in animal experimentation or testing purposes under prevailing legislation applicable to the research facility where such experimentation occurs.

The invention may be used in methods designed to assay putative modulators of weight, temperature regulation or behaviour. The animals of a test group will be provided with a diet which may be hypo, hyper or iso-caloric, and the weight gain or loss of the test group measured and compared to suitable control groups. Usually, test animals are provided with surplus food, the intake of which may then be determined by measuring the difference between the food provided and food remaining after a period of time.

The putative modulator may be any candidate substance which may be involved in regulation of weight, temperature or behaviour. For example, candidate substances include hormones or other peptides (including for example leptin, insulin, thyroid, hormone, TNF),(Huang, Q. Et al., Endocrinology 139, 1524-1532, 1998; Hwang, C. S. et al., Ann. Rev. Cell. Dev. Biol. 13,231-259,1997), prostaglandins, synthetic or naturally occurring chemical compounds, for example extracts of plants, steroids, benzodiazapenes, dexfluoroamphetamines or other amphetamine derivatives (Popovich, N. G. et al., J. Am. Pharm. Assoc. Wash., 37,31-39,1997). Compounds may be administered to test animals by any suitable route, for example orally or by intravenous injection, although other routes (e.g. buccal, nasal, transdermal, rectal, etc, are not excluded). The dose of a putative modulator will depend upon its nature and potency, and may be determined by those of skill in the art taking into account the nature of the test substance.

In a preferred aspect of the invention, test animals will be used in the assay of putative modulators of weight and/or thermoregulation. Modulators which affect the rate at which body weight is increased may be useful in treating obesity or weight related conditions such as diabetes. Modulators which restore proper thermoregulation, or alleviate the reduction in temperature, (Gong et al., J. Biol. Chem. 26, 24129-24132,1997) may be useful in treating conditions such as hypothermia or pyrexia of any origin, for example pyrexia due to endotoxin stimulus during infection, (Doig, G. S. et al. Crit. Care. Med. 25, 1956-1961, 1997) pyrexia associated with cell necrosis or hypespyrexia as a consequence of halothane or other drug administration (Kim, S. H. et al. J. Trauma 44, 485-491, 1998).

Additionally, the phenotypic effects on behaviour, particularly maternal behaviour, may be used as a determinator in the development of drug therapies for depression including post-natal depression or other behavioural disorders. The phenotypic effects may also be used in the development of therapies for modifying olfaction, cognition and male behaviour.

In a further aspect of the invention it has observed in the art that in the preoptic nucleus of the hypothalamus, significantly fewer oxytocin containing neurons are found in transgenic Peg3 knock-out animals as opposed to control animals. This suggests that either these neurons have never formed or that, importantly, they may have degenerated by one of several processes. PEG3 may therefore be important in the control of cell death or degeneration, particularly in the nervous system. Accordingly, the present invention provides transgenic animals of the invention in methods of testing compounds with the ability to enhance or repress degeneration of neurons in the brain or other tissues. Measuring the activity, amount or integrity of PEG3 may also act as a diagnostic or staging test for diseases involving cell death and degeneration, such as Alzheimers disease. Furthermore, compounds which alter cell degeneration via the PEG3 pathway may be used in methods of treatment of cancer.

Accordingly, in a further embodiment of the invention transgenic animals may be used in a method of testing the potential carcinogenicity of compounds by administering a test compound to a transgenic animal of the invention and determining whether said animal has an increased risk of tumour development compared to non-transgenic controls and/or untreated transgenic controls.

Animals of the invention may also be used as recipients of xenografts, particularly xenografts of human tumour cells. The efficacy of candidate anti-tumour compounds may then be tested in such transgenic animals to analyse the mode of action of the test compound and determine whether said mode of action is via a pathway regulated by or requiring PEG3.

The finding that PEG3 is associated with the phenotypic disorders mentioned herein allows the development of genetic markers to determine the susceptibility of an individual to obesity or other disorders. Thus in a further aspect of the invention, the PEG3 gene from obese subjects, including human subjects, may be analysed with nucleic acid of the invention, compared to the gene found in non-obese subjects. In the case of human subjects, obese individuals are preferably those with a body mass index (b.m.i., calculated as weight (kg) divided by height (m) squared) of over 25, and preferably over 30.

Nucleic acid—e.g. mRNA from cells expressing PEG3 or DNA from any cells may be analysed by any suitable means for detecting variation in individuals. Suitable methods include determining restriction fragment length polymorphisms (RFLPs), PCR product polymorphisms (i.e. the length of an amplified region of the gene produced using a primer pair), direct sequencing of all or part of the gene, or heteroduplex analysis. One or more of these methods may be used to determine features in the PEG3 gene of obese individuals which is associated with obesity. Similarly, associations with other disorders may be determined.

The association need not be one which is found in 100% of obese subjects and 0% of non-obese subjects. Predictive markers for phenotypic traits may be those which are found at a higher frequency in the subject population than in controls. Thus markers may be developed which indicate an increased risk of obesity or other PEG3-deficient-associated traits, so that individuals at risk may be treated before symptoms occur.

In an analogous manner, individuals showing symptoms of any of the disorders we have found to be associated with PEG3 inactivation may be screened for a polypeptide of the invention, for example using antibodies of the invention.

The provision of nucleic acid or protein markers of the PEG3 gene associated with obesity, temperature regulation, behavioural disorders, apoptosis, cell survival or resistance to infectious diseases provides a further aspect of the invention. Diagnostic methods and markers may be used on samples from individual subjects, to determine that individual's own risk of developing the one of these conditions, or the prognosis for treating the condition. For example, where particular polymorphisms such as deletions, truncations or substitutions of the wild-type PEG3 gene are found to be associated with one of these conditions, then a nucleic acid probe may be prepared to detect the presence or absence of the particular polymorphism.

Tests for detecting nucleic acid generally comprise bringing a human or animal body sample containing DNA or RNA into contact with a probe comprising a polynucleotide or primer of the invention under hybridizing conditions and detecting any duplex formed between the probe and nucleic acid in the sample. Such detection may be achieved using techniques such as PCR or by immobilizing the probe on a solid support, removing nucleic acid in the sample which is not hybridized to the probe, and then detecting nucleic acid which has hybridized to the probe. Alternatively, the sample nucleic acid may be immobilized on a solid support, and the amount of probe bound to such a support can be detected. Suitable assay methods of this any other formats can be found in for example WO89/03891 and WO90/13667.

Probes used in such techniques may be in the form of a short probe (for example of 15 to 50, such as 18 to 24 nucleotides) which is capable of hybridising to the wild-type sequence and not to the disease associated sequence, or vice versa. The probe may be packaged in a kit with suitable control reagents, instructions, and the like.

In another embodiment, the sample nucleic acid may be in the form of whole chromosomes, for example as a metaphase spread. A nucleic acid probe or primer of the invention may be labelled with a fluorescent label to detect the chromosomal location of a PEG3 gene in the spread.

The identification of a role for PEG3 in the above described manner provides a novel target for therapeutic agents. Modulators of PEG3 obtained by assay methods of the invention may be used to treat a variety of conditions, including, for example, depression, impaired maternal care, aberrant thermoregulation and obesity. The latter, in the form of late onset obesity, can occur with ageing, partly due to changes in energy balance and a lack of thermoregulation and thus Peg3 forms a target linking the two and may be critical to devising appropriate treatments for such conditions.

The identification of PEG3 alleles or polymorphisms in human populations associated with obesity will provide a means to identify individuals at risk of obesity at an early stage, and thus provide appropriate treatment or the introduction of life-style changes which may reduce the effect of a risk-associated allele or phenotype.

The invention is illustrated by the following examples.

RT-PCR and Cloning.

Placental cDNA was first denatured at 94° C. for 5 min and amplified with High Fidelity PCR system (Roche) for 30 cycles (94° C. for 30s, 58° C. for 1 min, 68° C. for 2 min) using primers HFL1 and HFL2 (synthesised by Genosys). The priming sequence of primer HFL1 was picked from the cDNA sequence ZIM2 (accession no. AF166122, nucleotides 368-389) while that of the primer HFL2 was designed according to the cDNA KIAA0287 (accession no. AB006625; nucleotides 147-168). An EcoRI linker sequence 5′-GATCGAATTC . . . . 3′ was added at the 5′ end of each primer (see SEQ ID NO.23 and 24) to facilitate cloning. The PCR products were precipitated with sodium acetate and ethanol. The pellets were then resuspended and digested with EcoRI at 37° C. for an hour (NEB). The digested PCR products were ligated to the EcoRI-digested pbluescript SK II +vector (Stratagene) in a 3:1 ratio using the Rapid Ligation System (Roche). The ligation products were mixed with the competent DH5α E. coli cells and the DNA-cells were heat-shocked at 42° C. for exactly 90s. The transformed cells were selected on IPTG+X-gal (isopropyl-βD-thiogalactopyranoside+5-bromo-4-chloro-3-indolyl-βD-thiogalactopyranoside)+ampicillin plates. The white colonies were picked and tested for the presence of inserts. The colonies were first lysed in 100μl of H₂O. 10 μl of the lysate was then PCR-amplified using the T3 and T7 primers as described above.

Sequencing.

Four clones containing the inserts were randomly picked for sequencing. The bacterial colonies for each clone were inoculated into 2×1.5-ml LB broth containing 100 μg/ml ampicillin and allowed to grow overnight at 37° C. Plasmid DNA was extracted from the culture using either alkaline lysis method (Maniatis et al., 1989. Molecular cloning) or Qiagen column according to the manufacturer's manual. The DNA was then sequenced in both direction using T3 and T7 primers at Department of Biochemistry, Cambridge, using the ABI system (Perkin Elmer).

Results.

The PCR products amplified by primers HFL1 and HFL2 were cloned into the pBluescript SKII+ vector at the EcoRI site. The recombinant plasmid was subsequently transformed into the DH5α E. coli cells as described. 30 out of 6000 colonies screened were white and 16 white colonies were picked and tested for the presence of inserts using PCR. All the colonies picked contained the inserts. Plasmid from four randomly chosen colonies was used for sequencing and the sequence of the clones were assembled and analysed using the software Sequencher™ 3.1.1. Three of the clones were sequenced in both directions and the fourth in one direction only. These sequences are shown as SEQ ID NOs:2-8.

We claim:

1. An isolated cDNA having the sequence of SEQ ID NO:1.

2. An isolated cDNA fragment of SEQ ID NO:1, said fragment being selected from the group SEQ ID NO:2-8.

3. An isolated cDNA having the sequence of SEQ ID NO:9.

4. An isolated cDNA having the sequence of SEQ ID NO:13.

5. An isolated cDNA encoding the polypeptide sequence of SEQ ID NO:10.

6. A method of expression of PEG3 protein, said method comprising expressing a nucleic acid encoding SEQ ID NO:9 operably linked to a promoter in a host cell compatible with said promoter.

7. A method of detection of PEG3 DNA or mRNA in a sample, said method comprising bringing said sample into contact with SEQ ID NO:1 under conditions which allow SEQ ID NO:1 to hybridise to a complementary sequence, and detecting whether or not hybridisation has occurred.

8. A method of detection of PEG3 DNA or mRNA in a sample, said method comprising bringing said sample into contact with a fragment of SEQ ID NO:1, said fragment comprising at least 15 nucleotide of SEQ ID NO:1, under conditions which allow SEQ ID NO:1 to hybridise to a complementary sequence, and detecting whether or not hybridisation has occurred.

9. A method for amplifying a portion of the PEG3 cDNA, said method comprising:
providing the primer pair of HFL1 (SEQ ID NO:23) and HFL2 (SEQ ID NO:24);
bringing said primer pair into contact with a sample of human cDNA;
performing a polymerase chain reaction; and
recovering said amplified portion of said PEG3.

10. The method of claim 9 which further comprises inserting said amplified portion into a vector.
